(19) United States
(12) Patent Application Publication
(10) Pub. No.: US 2010/0036207 A1
(43) Pub. Date: Feb. 11, 2010

(54) Relative response system including reprogramming capability for autonomous or interrelated stimulus and sensor systems for measuring biological response data relative to either an absolute reference and/or relative to other biological response

(76) Inventors: Michael Zack Eckblad, Auburn, WA (US); Ken DeMile, (US)

Correspondence Address: Michael Zack Eckblad, 36120 208th ave se, Auburn, WA, 98092, US

(21) Appl. No.: 12/460,750

(22) Filed: Jul. 24, 2009

Related U.S. Application Data
(60) Provisional application No. 61/135,779, filed on Jul. 24, 2008.

(51) Int. Cl. A61B 5/16; A61B 5/00

(52) U.S. Cl. 600/300

(57) ABSTRACT

A wireless system of independent CPU pods for initiating, collecting, and optionally processing, absolute and/or relative response data of biological systems to stimulus and then using wireless means to transmit the data to a plurality of external communications (e.g. human interface device) via a bridge pod. The system is set up to perform a plurality of sequences of stimulus and then response measurement according to programming through the wireless network which can be changed on each CPU pod via re-programming at any time to create new configurations and metrics. The sequences may execute on each CPU pod(s) and CPU bridge pod (s) autonomously or the programing can create a plurality of different interrelated sequences depending on system objectives. A plurality of sensors and stimulate devices are connected or integrated to the CPU pods according to the purposes of the programmed stimulate response sequences. This system is beneficial for getting stimulus-response data for any biological activity in a relative of independant response manner making it quite general. Thus, interrelated reactions of any number of biological systems relative to each other can be measured, processed and communicated to any desired device for further processing or display. Thus, either individual performance or entire teams relative performance can be tracked to improve performance in a plurality of ways in a plurality of sports, military, etc. exercises.

### RELATED APPLICATIONS

The present application is a continuation application of United States provisional patent application, Ser. No. OC000000031445831, filed Jul. 24, 2008, for COLLECTING, AND OPTIONALLY PROCESSING, REACTION DATA OF BIOLOGICAL SYSTEMS TO STIMULUS DATA OR TO RELATIVE MOVEMENTS AND TIMINGS BETWEEN THEMSELVES (APP #61/135,779; CONF #6561), by Ken DeMile, Michael Zack Eckblad, included by reference herein and for which benefit of the priority date is hereby claimed.

This invention is unique in that it uses wireless networking technology, independant programming and reprogramming of a plurality of autonomous microprocessor based CPU pods to setup, initiate and measure a sequence of events involving a one to a plurality of biological systems relative to each other and/or relative an absolute reference. Any of the autonomous microprocessor CPU pods can command, initiate, and sense stimulus-response events at any time effectively making a system whose characteristic function can be changed at any time with simple re-programming. Because of the ability to re-program the CPU pods, the systems provide general accommodation of any sensor or stimulus device. Simple single-event-single-measure systems such as punch in response to a light or counting number of punches in response to light, fail to provide the flexibility to measure multiple action of multiple biological systems against each other and/or relative to an absolute time reference. Further, existing systems do not allow for re-programming the complete character and relative measuring capabilities at any point in time as part of the normal system function. Rather, other applications are software or hardware hard-coded as well as dependant on hard wired system from the start to perform one characteristic function. This hard coding of other applications dictates a specific stimulus and sensor hardware configuration as well.

### FIELD OF THE INVENTION

The present invention relates to reaction data collection of biological systems to stimulus using microprocessors, sensor, and, more particularly, to using independant CPU pods on a wireless network for sensing and processing sequences for measuring biological response to stimulus in a plurality of configurations via re-programming in an absolute and/or relative reference.

### BACKGROUND OF THE INVENTION

Measuring biological reaction to stimulus is currently problem specific, difficult or impossible to reconfigure, and has wired data transmission interconnectivity that interferes with movement. These limitations restrict the ability of coaches, scientists and individuals to understand and optimize the performance of biological systems in a general way whether it be response of a cow to an electric fence or response of a boxer to a light or a punch coming his way or relative reactions between a throws and catches on a baseball team. This invention seeks to enable a system that can accommodate a plurality of sensors and stimulus devices and can be reconfigured to make any combination of stimulus-response with a simple standard upload of programming software. Further, wires should be eliminated at the network level.

There are currently some attempts at general study of biological systems in existence. We start with some general examples of products and special application efforts.

First is the homemade approach as typified by these quotes from http://www.physicsforums.com/archive/index.php/t-173340.html:

“I was wondering i anyone could help me. I am trying to measure the speed of a karate punch in order to calculate the force of the strike. I have read several papers on the subject but each of these use a high speed video camera and software such a video to analyze the data. Does anyone know of another way to measure the acceleration? I only have a standard camera at 30 frames per second?”

“Measure the total impulse, by striking a heavy bag and seeing how high it swings.”

“If you or one of your friends knows a bit about electronics, it should be quite simple to build a chronograph. Use a couple of strips of aluminum foil a set distance apart as your triggers and punch through them. When you break the first one, the timer starts. The second one stops it.”

The commercial type is illustrated by the “HitMaster” (http: //impacttrainingsystems.com/HitMaster.html) which uses various signals and then measures the humans time to strike a target in response. It will also count the number of hits in a period of time. All data is local to the workout station and all communication is with wired connections. Another type is typified by the Nike or Adidas foot pod which wirelessly sends data to a watch or handheld device to be observed by the user.

Finally there is the custom research approach such as seen on “Sports Science” television program, large company, or university study which has custom built sensors, computers, interconnectivity, software, and supporting scientists. These studies are designed to collect data for a specific problem and use it for research or entertainment.

This is a list of patents that have similarities:

U.S. Pat. Nos. 6,002,336, 4,941,660, 6,056,674, 4,534,557

The homemade method suffers from the issue of being non-standardized and high variability of accuracy. It frequently avoids any complexities such as wireless or general sensor interface. Thus it is useful only to end user and his perception of value.

The current commercial versions are centered on a particular application such as boxing or running. Mostly they are wire-connected solutions. Further they tend to be centered on a single station and are not scalable to a whole set of stations able to communicate with all the other stations or produce interrelated relative response data among several interrelated biological systems. Further, they lack the flexibility to be re-programmed at anytime to perform a completely different function and to accommodate any type of sensor and stimulus hardware configurations not previously considered in the initial design.

The research solution, despite its possible depth and accuracy, is not usable or affordable by most people or even companies. It is a point solution, custom developed to meet a specific need for data, not a specific solution that is affordable for the average individual or company.

Other patented stimulus-response systems are problem specific and lack flexibility to be reconfigured or measure relative response of interrelated systems as discussed in the commercial version.

### SUMMARY OF THE INVENTION

In accordance with the present invention, there is provided a wireless system of autonomous, independent CPU pods for initiating, collecting, and optionally processing, response of one or a plurality of biological systems to stimulus and then using wireless means to transmit the data to a plurality of external communications (such as a notebook or Internet) via a CPU bridge pod and/or to other CPU pods. The system is set up to perform a plurality of re-programmable sequences of stimulus and then response measurement. The biological response data sensor(s) reference may be relative to an absolute reference and/or relative to each other. A plurality of sensors and stimulus devices are connected with the pods according to the purposes of the re-programmable sequences.

It would be advantageous to provide a system of wireless data collection CPU pod(s) that can be re-programmed at any time with behavior by a plurality of external communications such as a computer.

It would also be advantageous to provide a solution that is scalable from one to a plurality of CPU pods optionally capable of signal processing that can have a plurality of stimulus or sensor devices attached and can communicate to each other independently or all CPU pods simultaneously.

It would also be advantageous to provide a system of independent communicating CPU pods that can be located in close proximity (touching) or at far proximity (miles) while still providing the ability to synchronously or asynchronously collect biological response data to stimulus.

It would be advantageous to use the same CPU pod(s) for both initiating stimulus, optionally signal processing, and/or measuring response and then sending the data to the external communications or to each other.

It would be advantageous to provide a general input and output capability at each CPU pod so that the nodes can be re-programmed at any time to have whatever behavior is desired as a normal operating procedure.

It would further be advantageous to have the relative reaction time of each of a plurality of biological responses referenced to either an absolute clock and/or relative to each other.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a plan view of a FIG. 1 is a plan view of a system for gathering time based stimulus response data of biological systems using a multiple autonomous synchronized sensor systems;

FIG. 2 is a plan view of a FIG. 2 is a plan view illustrating an application of relative reaction sensor to a human and a punch bag acting and reacting while synchronized time and force data is acquired; and

FIG. 3 is a detail view of a FIG. 3 is a detail view of the basic functions of the relative reaction sensor and analysis system.

For purposes of clarity and brevity, like elements and components will bear the same designations and numbering throughout the Figures.

### DESCRIPTION OF THE PREFERRED EMBODIMENT

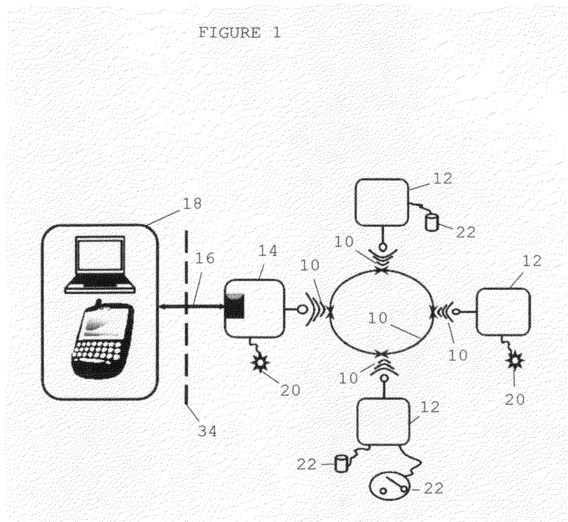

FIG. 1 is a front plan view of an integrated system of components that initiate and collect biological response data either asynchronously or in a synchronized manner according to programmed stimulus response sequence 28 established within the various CPU pod 12(s) in the system. The data is then processed and displayed in a plurality of ways.

### DETAILED DESCRIPTION OF COMPONENTS

The system starts with a wireless network 10 of any of a plurality of standards or topologies including all forms of Electromagnetic Frequency (EMF) or physical signaling including audio, as long as they are capable of transmitting data in or out of CPU pod 12 and CPU bridge pod 14. The wireless network 10 replaces the typical wires used to transmit data in other response measuring systems for biological systems.

The key components hanging on the wireless network 10 are 0 or more CPU pod 12(s) and 0 or more CPU bridge pod 14.

The CPU pod 12 may gather data from 0 or a plurality of sensor devices including switches, accelerometers, heart rate monitor, GPS, etc. If there are 0 sensor devices, the CPU pod 12 may simply act as a repeater for the wireless network 10. The CPU pod 12 has at a minimum a microprocessor and wireless communication hardware and any of a plurality of programming depending on the desired behavior for the CPU pod 12 and may or may not have means of data acquisition storage. Optionally, the CPU pod 12(s) can signal condition a plurality of sensor device signals, and then provide the data to the any of the CPU bridge pod 14(s) or any of the other CPU pod 12(s). Any of the pods may be used to initiate a plurality of synchronous or asynchronous data collection sequence with all or a subset of the other CPU pod 12 or CPU bridge pod 14. The CPU pod 12 and CPU bridge pod 14 drives or reads data to and from the attached stimulate device 20(s) or response sensor 22(s) respectively. Optionally, the CPU pod 12 integrate a plurality of sensors directly in the CPU pod 12 including GPS location and altitude data, heart rate data, blood pressure, body temperature, accelerometer, etc. The CPU pod 12 may also include a means to communicate with a human which may include a plurality of input or output human interface devices.

The CPU bridge pod 14 may be identical to the CPU pod 12 except it adds an interface for an external communication path 16 to allow data transfer to and from a primary external communications 18. There can be anywhere from 0 to a plurality of CPU bridge pod 14(s) in the system. For example, system can have 0 CPU bridge pod 14(s) when it operates independently of external communications 18. For example, the system could be set up by a human on a notebook (external communications 18) to have one CPU pod 12 turn on a light (stimulate device 20) in 5 minutes and send a signal for another CPU pod 12 to sense 1 minute of velocity data (sensor device) and store it for later use on the CPU pod 12 after the notebook and CPU bridge pod 14 is disconnected. Later, the CPU bridge pod 14 may be reconnected to the wireless network 10 to collect stored data or re-program stimulus response sequence 28. The CPU bridge pod 14 may be involved but is not a requirement for the system to conduct the actual execution of the stimulus response sequence 28.

The external communication path 16 provides a means for the CPU bridge pod 14 to communicate outside the wireless network 10 to an external communications 18. A plurality of methods and standards can be used including a wired connection, Bluetooth, or another network such as the Internet or a local area network. For example, the external communications 18 could be co-located with the CPU bridge pod 14 and communicate directly via a Ethernet and a CAT5 cable to a local network or directly to an integrated display or human input device. Another example, the CPU bridge pod 14 resides on the East coast of the USA and a Smart Phone (external communications 18) on the west coast uses the Internet to connect to the CPU bridge pod 14 via a server integrated into the CPU bridge pod 14 to program the CPU pod 12(s) or CPU bridge pod 14 to have a particular behavior and then uses the same connection to initiate and collect the data for a stimulus response sequence 28.

The external communications 18 is a plurality of devices including PC computer, Internet devices, laptop, PDA (Personal Data Assistant), Smart Phone, etc. that allow a human to input or sense output information from the system. Other forms include non-integrated inputs and outputs such as a separate keyboard and separate LED or LCD display that simply connects directly to the CPU bridge pod 14 to input and/or display output.

The stimulate device 20 is used to cause the biological system 24 to respond either voluntarily or involuntarily. The stimulate device 20 could stimulate any of the senses or directly drive the nerve system. An example of a stimulate device 20 is a light that turns on to cause a human to throw a punch. Another example is a heat element that touches a dogs leg and initiates a movement response in the dog when turned on. Another example is a neuro-electric device that sends signals directly to a biological systems nerve system.

The response sensor 22 translates biological responses into signal useable by the CPU pod 12. The signals are then used by the CPU pod 12(s) to sense the state of the biological response. For instance, a switch (response sensor 22) that closes (change state) when struck. Another example would be an accelerometer (response sensor 22) on a human track runner that records acceleration over a period of time (time history). Another example would be a nerve impulse sensor that senses nerve system responses.

How the Integrated System Works

The integrated system provides a plurality of configurations. Part of the reason for this is that the CPU pod 12(s) are programmable to a plurality of behaviors at any time. This behavior may be completely independent and dependant relative to any other CPU pod 12. Thus, the system is not tied to any particular sport or activity or even a specific biological system 24.

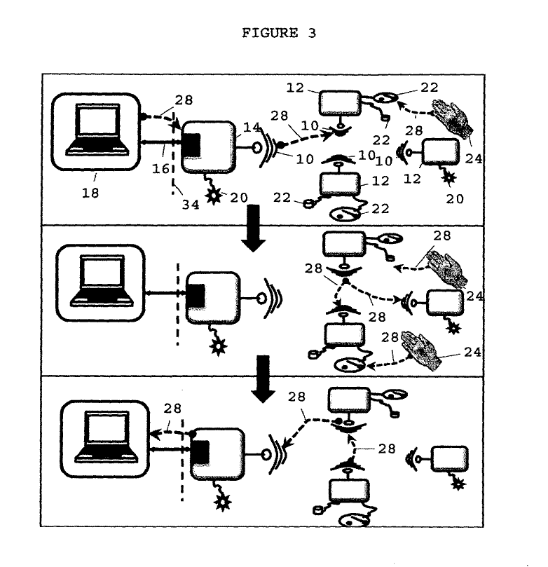

FIG. 3 shows the basic steps to a plurality of possible programming sequences. First, the programming which will support the desired stimulus response sequence 28 is pushed across the wireless network boundary 34 via external communications 18 path to each CPU pod 12 and CPU bridge pod 14 from the external communications 18. Second, the sequence is initiated from any of a plurality of indications such as specified time, random time, or a particular state of a sensor and signals via the wireless network 10 all involved CPU pod 12 (s) that the event has started and to initialize the start time. Third, any of a plurality of stimulus device events are executed according to the stimulus response sequence 28 and biological system 24 responses (both immediate and consequential) are recorded and optionally signal processed. The stimulus response sequence 28 may include dependant sequential initiations of biological systems relative to each other. For instance a pitcher biological system 24 may throw the ball after a light stimulate device 20 initiates response while later in the stimulus response sequence 28, the first base biological system 24 timing starts after he catches the ball for an initiation of response. Finally, the results are reported to other CPU pod 12(s), CPU bridge pod 14(s), and/or external communications 18 for any of a plurality of uses including sensory readout (e.g. visual display), further processing, storage, and/or retransmittal.

Thus, system creates response sensor 22 input based sequences via CPU pod 12 and CPU bridge pod 14 programming that measure the response of a biological system 24 by creating a stimulus and measuring a response as the sequences are executed. This creates a system adaptable to many types of study including athletic, military, animal, and medical. Two examples will be shown here.

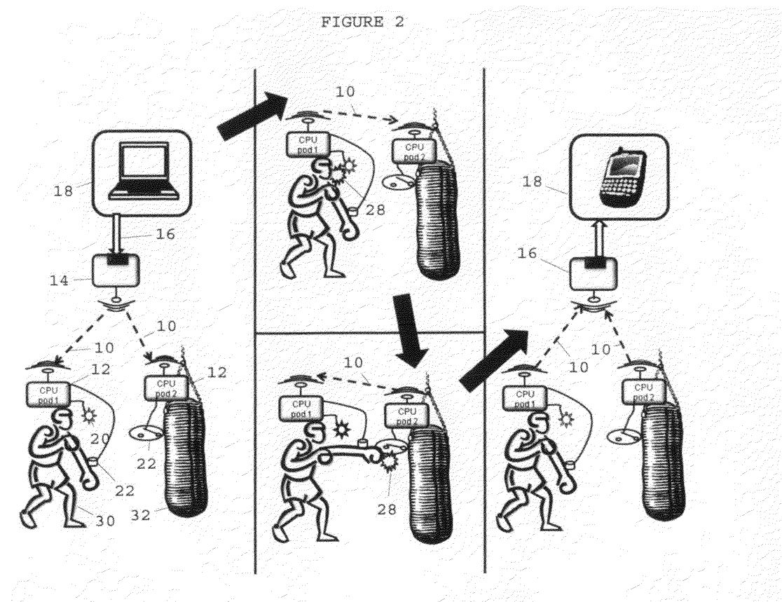

FIG. 2 is a plan view which adds an example of interaction with some typical biological unit; an athlete 30 and a punch bag 32. In this example, a human and a punch bag 32 each have a CPU pod 12 attached in any of a plurality of methods. A programmed stimulus response sequence 28 of events, scheduled to start at time 0, is sent as programming from a external communications 18 (e.g. laptop) to the CPU bridge pod 14 and then specific sequences are sent individually to the CPUpod 1 and CPUpod 2. At time 0 a stimulation device (light) is turned on, and CPUpod 1 communicates via wireless network 10 a start timing command to all involved CPU pod 12(s) (CPUpod 1). The athlete 30 then hits the punch bag 32 as soon as possible to stop the timer via a switch response device on the punch bag 32 monitored by CPU pod 12 2. At the same time, location and motion of the athletes hand is tracked by an accelerometer response device attached to the wrist and fed into CPUpod 1 until CPUpod 2 indicates, via wireless network 10, impact and first location data via double integration until the first hits the response device switch mounted to the punch bag 32. Part of the data is stored on the CPUpod 1 and part of it on CPUpod 2 all synchronized to a common relative time. After the sequence is complete, CPUpod 1 and CPUpod 2 send the data to the CPU bridge pod 14 to be post processed and then displayed on the external communications 18. As example of reprogramming, a new sequence of events could then be sent over the external communications 18 to cause the light to flash quickly via a new program on CPU pod 12 1. At the same time new stimulus response sequence 28 programming is placed on the CPU pod 12 2 causing it to read time and number of punches per second. After the athlete 30 has completed this sequence, the data from CPU pod 12 2 (due to the new programming) stores the data on the onboard memory and then at a specified time, sends the data via CPU bridge pod 14 and external communications 18 over the Internet to a specified University Internet address for data analysis and display for qualification assessment for the boxer via independent experts at the university. As an example of relative response, a second boxer and punch bag 32 with equivalent CPU pod 12(s) could be set up 6 ft from the original. The second boxer sequence does not start from time 0 but rather relative to after the first boxer hits his punch bag 32 switch response sensor 22 which is when timing starts for the second boxer. This important relative response function illustrates how an entire teams relative performance can be tracked to improve performance in a plurality of ways in a plurality of sports, military, etc. performance exercises.

Another example showing non-locality of the external communications 18. The system could be set up by a human on a smart phone over the Internet (external communications 18) from one side of town to the wireless network 10 on the other side of town; having one CPU pod 12 turn on a light (stimulate device 20) and send a signal for another CPU pod 12 located on a human to sense heart rate data (sensor device) and send each reading back to the smart phone over the Internet (external communications 18) to be plotted out as each point becomes available. At any time the CPU pod 12 in this example can be reprogrammed or the function changed to an entirely different CPU pod 12 or CPU bridge pod 14 allowing a plurality of configurations with the same CPU pod 12 and CPU bridge pod 14 set.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A relative response system including reprogramming capability for autonomous or interrelated stimulus and sensor systems for measuring biological response data relative to either an absolute reference and/or relative to other biological response for collecting, and optionally processing, stimulus-response data of biological systems, comprising:
a wireless network, for providing data transmission between cpu pod(s) and cpu bridge pod(s) and also, when needed, a transmission path for software reprogramming of cpu pod(s) and cpu bridge pod(s);
an integrated wireless negotiation and microprocessor, power source, independently programmable and reprogrammable at any time cpu pod, for executing sequences of stimulus-response according to changeable programming either synchronously or asynchronously and with autonomous or interrelated behavior with other response sensors, other cpu pod(s) or other cpu bridge pod(s) and communicating on the wireless network;
a cpu bridge pod, for communication connection from wireless network to the external communication path as well as perform all functions that a cpu pod performs, wirelessly connected to said CPU pod;
an external communication path, for download and upload of programming and/or data between the cpu bridge pod and a plurality of external communications (e.g. network, computer, human, etc), electrically connected to said CPU bridge pod;
a stimulus response sequence, for software coded instructions for executing a plurality independent and/or interrelated stimulus-response actions for an individual biological system or a group of biological systems in order to measure a plurality of biological response data relative to an absolute reference and/or a reference of one response sensor relative to another;
a stimulate device, for stimulating any aspect of a biological system including anything a biological system can sense voluntarily or involuntarily, electrically connected to said CPU pod; and
a response sensor, for sensing any aspect of a biological system, electrically connected to said CPU pod.
